(19) United States
(12) Patent Application Publication
(10) Pub. No.: US 2018/0259441 A1
(43) Pub. Date: Sep. 13, 2018

(54) OCT Sensing of Particulates in Oil

(71) Applicant: Axsun Technologies, Inc., Billerica, MA (US)

(72) Inventors: Bartley C. Johnson, North Andover, MA (US); Joseph Paul Little, III, Austin, TX (US); Robert K. Jenner, Lowell, MA (US)

(21) Appl. No.: 15/914,221

(22) Filed: Mar. 7, 2018

Related U.S. Application Data
(60) Provisional application No. 62/468,180, filed on Mar. 7, 2017.

(51) Int. Cl. G01N 15/14; G01N 15/06; G01N 15/02; G01N 21/45; G01N 33/28

(52) U.S. Cl. CPC G01N 15/1434; G01N 15/06; G01N 15/0205; G01N 2015/1075; G01N 33/2835; G01N 2015/0693; G01N 21/45

(57) ABSTRACT

A fluid sample is analysed in-line by OCT techniques. The density, size, velocity and other attributes of particles present in a fluid, oil, for example, that is flowing through a conduit.

### RELATED APPLICATIONS

This application claims the benefit under 35 USC 119(e) of U.S. Provisional Application No. 62/468,180, filed on Mar. 7, 2017, which is incorporated herein by reference in its entirety.

### BACKGROUND OF THE INVENTION

Optical coherence tomography (OCT) is an imaging technique that uses coherent light to capture micrometer-resolution, two- and three-dimensional images from within optical scattering media, biological tissues, for example.

Generally, OCT techniques involve an interferometry arrangement that sends light into both a sample and a reference arm of an interferometer (e.g., Michelson or Mach-Zehnder). The signal is received by a detector or line-scan camera, and the digitized signal is processed. An image of the target is created by the interference pattern.

### SUMMARY OF THE INVENTION

The invention generally relates to OCT techniques and systems for obtaining information on particulates (solid, immiscible droplets, gas bubbles, etc.) present in a fluid, oil for instance, In specific embodiments, an OCT oil analysis is conducted on a moving stream, e.g., as oil flows through a pipe or cell. Characteristics that could be determined include particle density, particle flow velocity through Doppler methods and image processing of particle streaks, and/or particle size. Additional data such as index of refraction, images of water and air bubbles (and possibly other liquids), particle cohesions in water, flow velocities, optical transmission intensity, linked to particulate scattering and to refraction of non-uniform, e.g., immiscible, liquids also can be obtained in some cases.

Practicing the invention can present many advantages. Approaches described herein, for example, can provide data on particulates in crude and refined oil products, such as particle density, particle flow velocity, image processing of particle streaks, Doppler methods, particle size. Additional data that can be obtained relates to index of refraction of oil, images of water and/or air bubbles (and possibly other liquids). In some implementations, the optical transmission intensity is linked to particulate scatted ng and/or the refraction of different liquids.

Importantly, aspects of the invention can be practiced on samples that flow through a conduit, making possible “in-line” analyses. Techniques described herein can be used to detect particles types that are particularly relevant to the oil industry, asphaltenes and/or paraffins, for instance. In some cases, debris or “dirt” present in the oil also can be detected. Results obtained can then be used to assess potential clogging conditions, allowing operators to take measures for preventing or circumventing them. Importantly, the particles can be easily seen even at low signal to noise (SNR) ratios.

Versatile and robust, approaches described herein can be used in systems and applications found outside the oil industry.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is a schematic diagram of a reflection OCT arrangement for detecting particles in oil.

FIG. 2 is a schematic diagram of a transmission OCT arrangement for detecting particles in oil.

FIG. 3 is a plot of absorbance versus wavelength for an illustrative oil sample using embodiments of the invention.

FIG. 4 is an image of crude oil pumped through a windowed cell and monitored by a reflection OCT system such as shown in FIG. 1. At left is the annotated version of the image and at right the contrast enhanced version.

FIG. 5 presents side by side particle images obtained according to embodiments of the invention at different flow rates.

FIG. 6 is an illustration of the data processing steps that can be used to determine flow velocity by image processing.

FIG. 7A shows a calculation of flow velocity from FFT fit of FIG. 6c and the beam spot size.

FIG. 7B shows the equations relating to the Gaussian Fourier Transform pair.

FIG. 8 is a diagram showing side by side coaxial (left) and offset (right) probe designs for both reflection and transmission.

FIG. 9 is a schematic diagram of an arrangement for practicing embodiments of the invention.

FIG. 10 shows how the beam is focused in a sapphire flow cell relative to a cell for which n (index of refraction) is 1.

FIG. 11 is a series of images showing pump off and pump on conditions.

FIGS. 12 and 13 are further examples of data processing steps used to determine particle velocities by image processing.

FIGS. 14 shows particle images at two different flow rates.

FIGS. 15 through 18 show additional side by side particle images obtained at different flow rates.

FIGS. 19 through 21 show refractive index estimates for water in oil systems.

FIGS. 22 and 23 show series of images of samples of oil containing water particles (droplets).

FIG. 24 is a plot of the Doppler shift versus time for a milk-water system.

FIG. 25 presents an example of a Zemax Gaussian beam analysis.

### DETAILED DESCRIPTION OF THE PREFERRED EMBODIMENTS

The above and other features of the invention including various details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

As used herein, the term “and/or” includes any and all combinations of one or more of the associated listed items. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

In many of its aspects, the invention relates to detecting particulates in oil or another turbid fluid (e.g., a turbid liquid) such as milk. As used herein, the term “particulates” or “particles” refers to solid particles, liquid droplets, gas bubbles and the like. The oil can be animal, vegetable, or a petrochemical oil. in terms of petrochemicals, the oil can be unprocessed crude oil and/or a petroleum product that could be made up of refined crude oil.

Two particle types that are especially relevant to the oil industry are asphaltene (defined operationally as the n-heptane (C7H16)-insoluble, toluene (C6H5CH3)-soluble component of a carbonaceous material such as crude oil, bitumen, or coal) and paraffin (flammable, whitish, translucent, waxy solids including a mixture of saturated hydrocarbons, obtained by distillation from petroleum or shale). Tracking these and similar particulates is important to prevent clogging of refinery equipment.

in some embodiments, approaches described herein can be used to detect debris (e.g., dirt), often of unspecified origins. in other embodiments, the analysis is used on samples in which the oil contains water droplets. Further embodiments relate to a milk carrier.

An important feature characterizing some aspects of the invention is the “in-line” capability. As further described below, the invention can be practiced on flowing (moving) samples.

Particle detection can he conducted using a reflection and/or a transmission OCT arrangement.

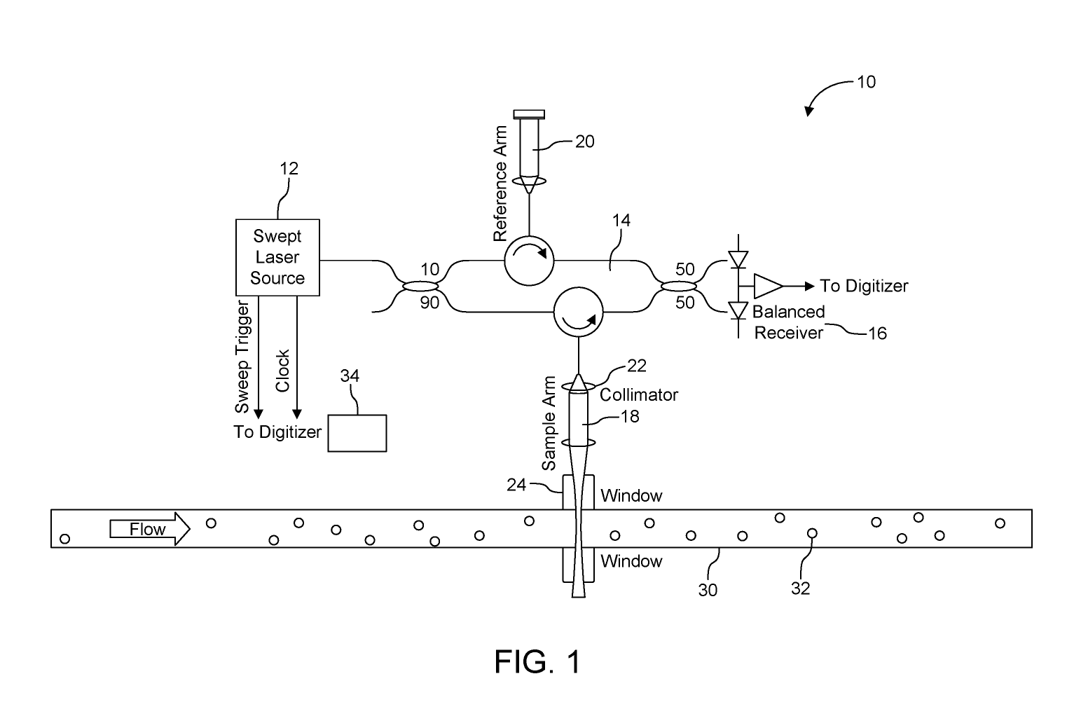

Shown in FIG. 1, for example, is reflection OCT system 10, including a swept laser source, e.g., tunable laser 12, interferometer 14 and balanced receiver 16, from which output is passed on to a digitizer. Also included are sample arm 18 and reference arm 20, collimator 22 (e.g., f=50 mm) and window 24. The material being analyzed is flowing fluid 30, containing particles 32. In specific embodiments, the fluid is an oil such as described above.

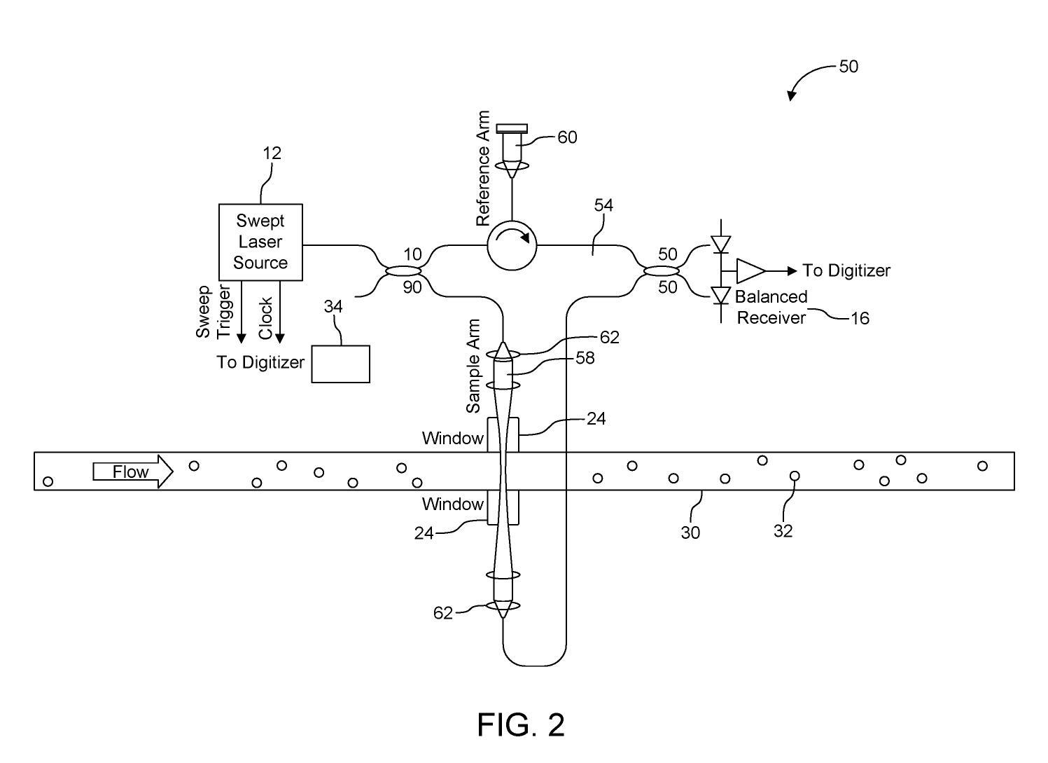

Shown in FIG. 2 is transmission OCT system 50, including a swept laser source, e.g., tunable laser 12, interferometer 54, and balanced receiver 16. Also included are sample arm 58 and reference arm 60, collimators 62 and 64 and window 24. As in the case of the reflection system above, the material being analyzed is a flowing fluid (e.g., oil) 30 containing particles 32.

Typically, the swept source can be provided with a sweep trigger and clock and is connected to a digitizer. While the examples in FIGS, 1 and 2 show a swept source type of setup, spectral domain and time domain analogs could be implemented as well.

Furthermore, the reflection and transmission (shown separately in FIGS. 1 and 2) can be performed simultaneously by splitting the laser power into two interferometers and utilizing a two-channel digitizer.

If desired, a galvanometer setup could be added to provide a spatial scanning mechanism.

In practice, images are formed by repeatedly imaging one beam line (A-line in the literature) as the oil flows by. Flow rates can be determined based on the length in time of the particle streaks. Typically, the longer the track in time, the slower the flow. Some embodiments rely on OCT Doppler methods to measure flow velocities.

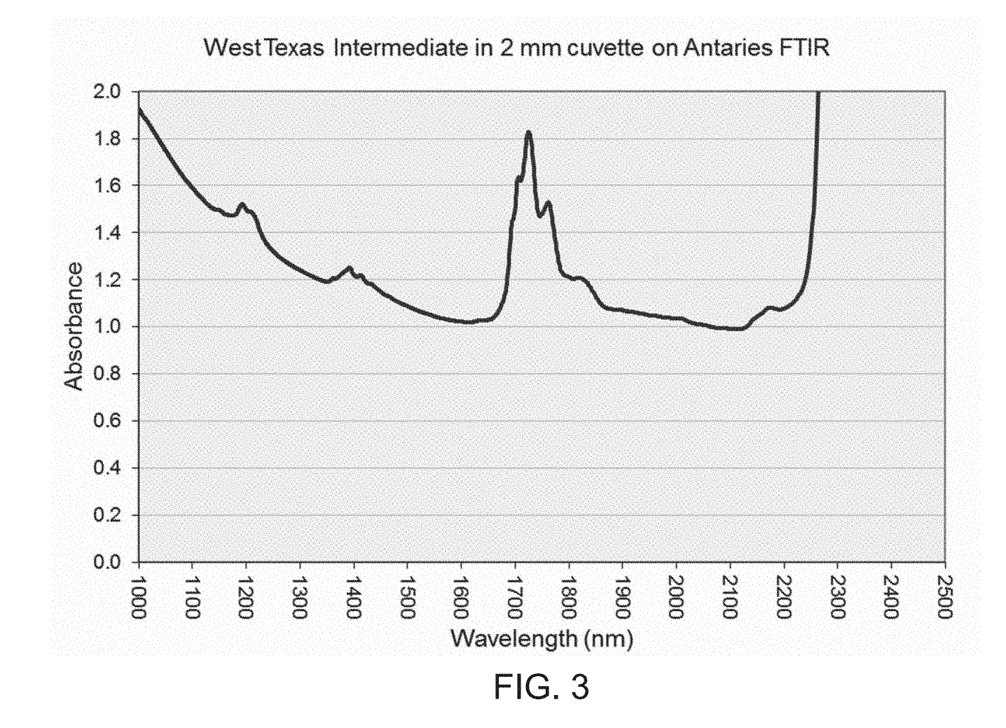

Shown in FIG. 3 is data obtained for an oil sample (West Texas intermediate) measured in the 1310 nm band using a 2 mm cuvette on an Antaries FTIR. The OCT data was taken in the 1300 nm band, but other suitable wavelength regions can be selected. In many cases, wavelengths with low oil absorption are preferred (FIG. 3).

The OCT data was taken with a 50 KHz A-line rate; the number of A-lines in the B-scan was 500; and the total sample time was 10 milliseconds (ms)

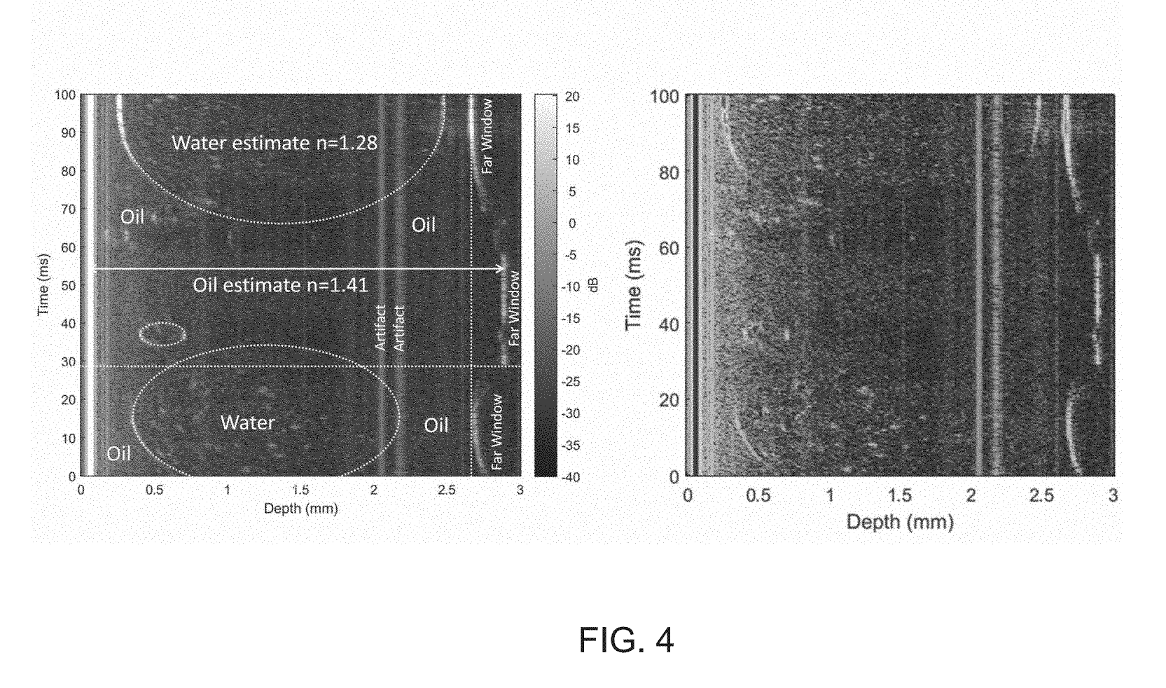

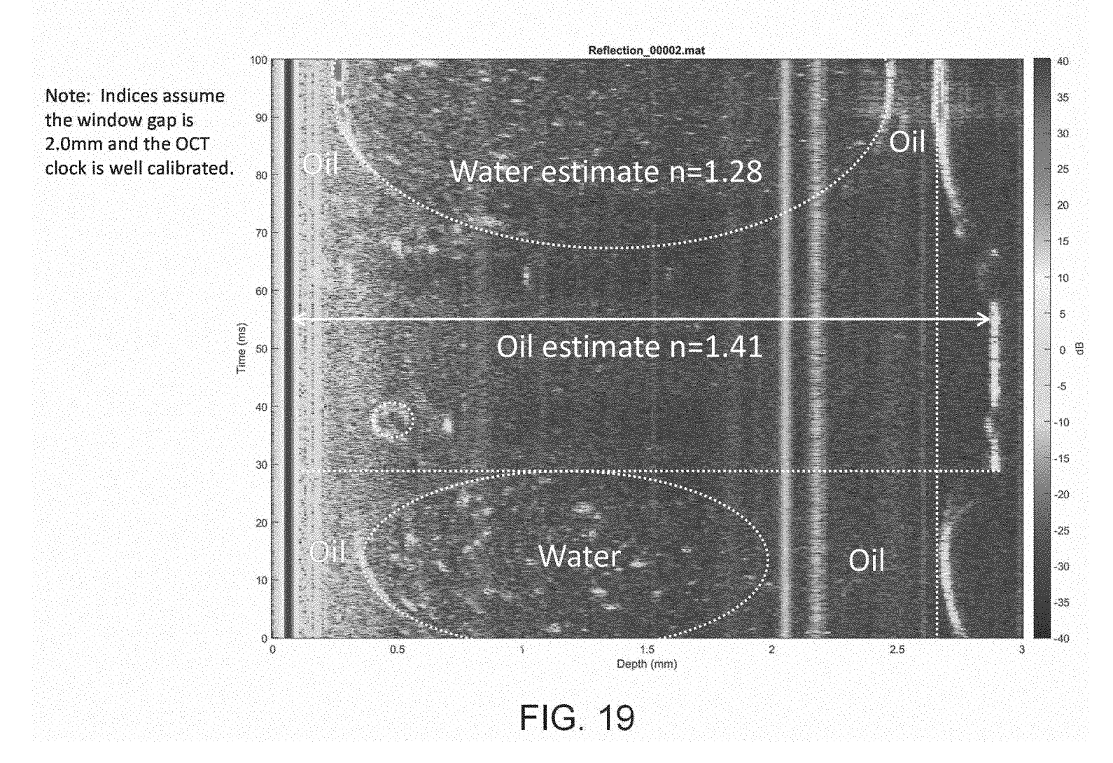

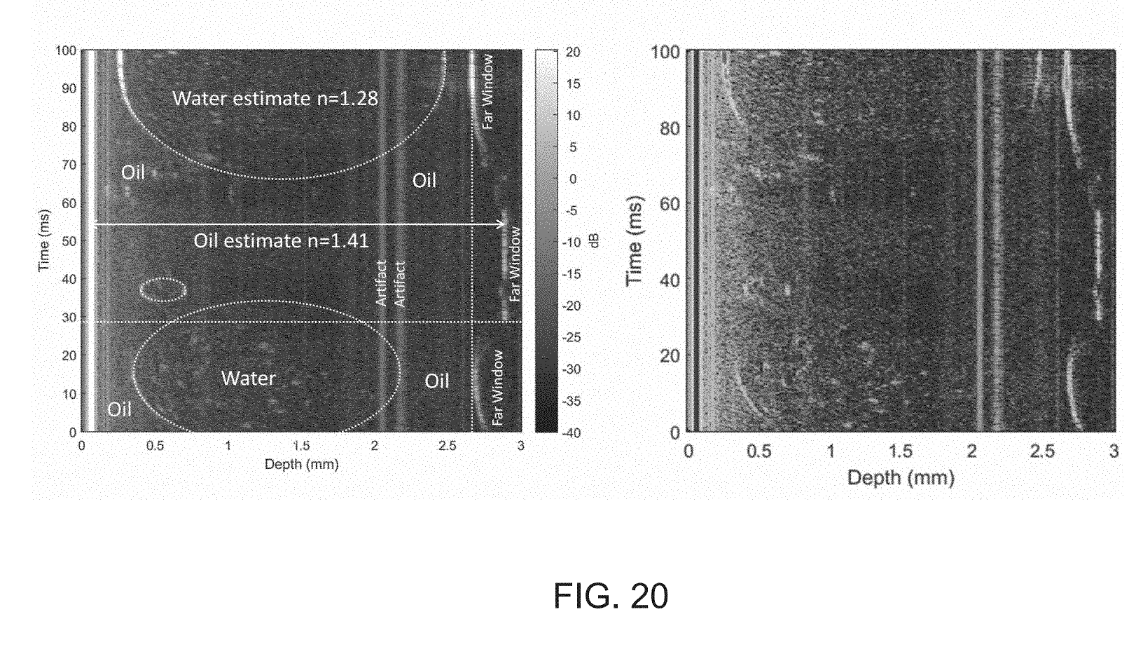

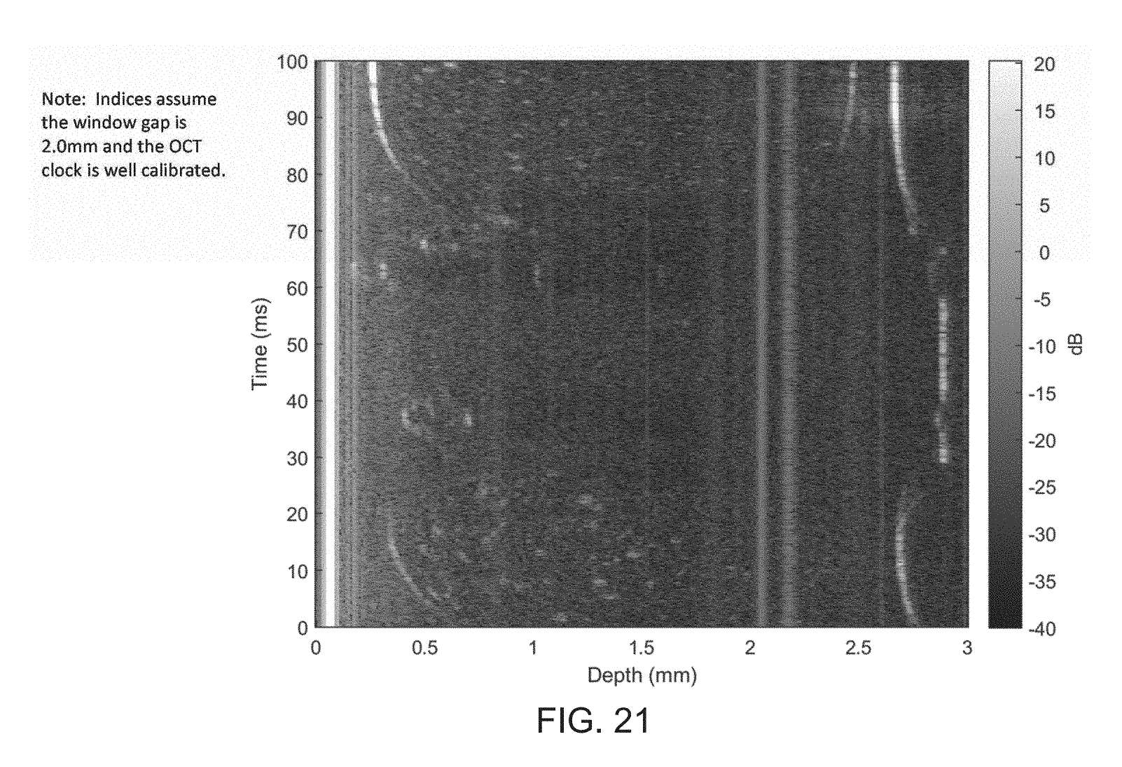

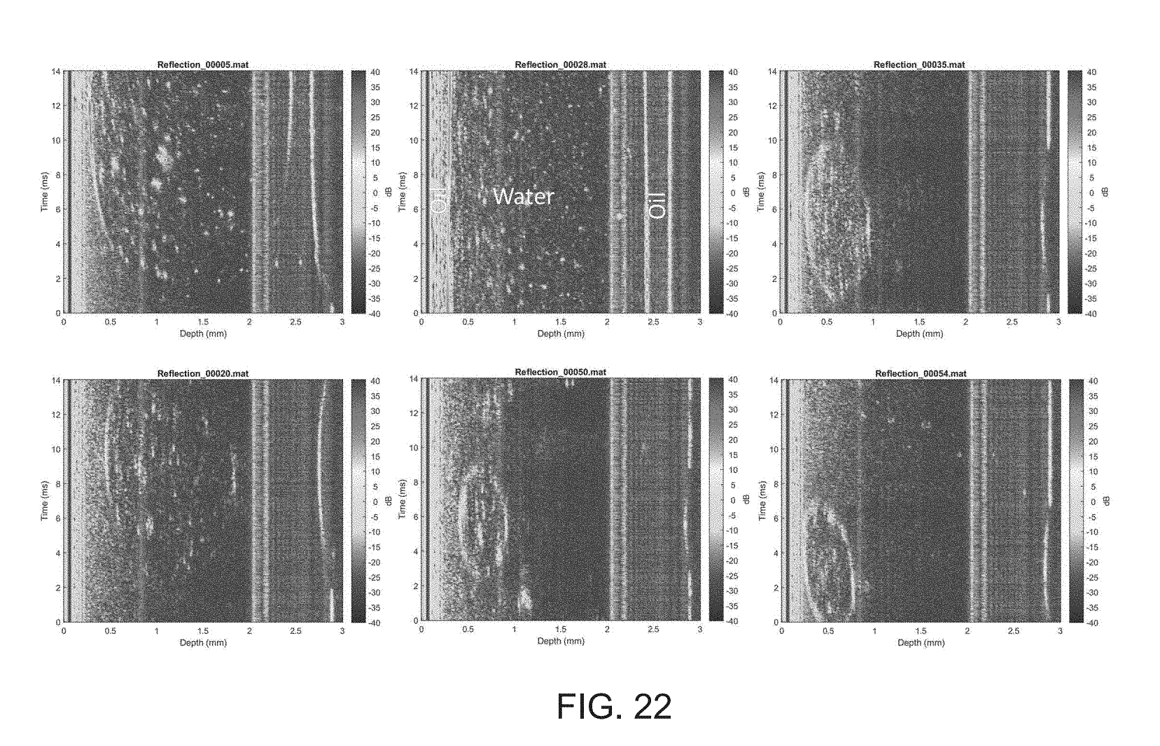

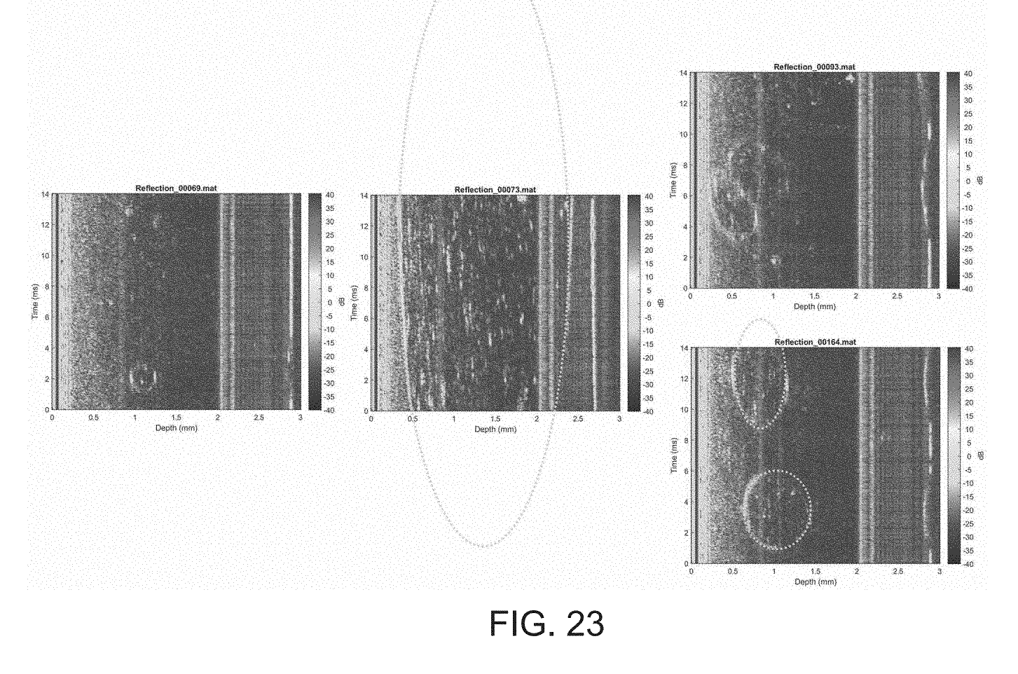

Shown in FIG. 4 are two versions or presentations (annotated at the left and contrast enhanced at the right) of the same image of crude oil and water pumped through a windowed cell and monitored by reflection OCT. The image was obtained with a system such as that of FIG. 1. The index of refraction (n) of water at 1300 nm is approximately 1.323; the approximate n of oil was estimated as 1.41. Refractive index estimates are illustrated in FIGS. 19 through 21, for example. Additional images of oil samples containing water droplets are shown in FIGS. 22 and 23.

The image in FIG. 4 demonstrates the utility of the reflection OCT measurement, as oval water bubbles can clearly be seen. The images also show that the water attracts particles, pulling them out of the oil. Another telltale sign of the water is the refractive index difference. The horizontal axis, marked “Depth”, is actually the “Optical Depth”, which is the refractive index multiplied by the physical depth. Reflections from both the near and far windows can be seen in the image. The physical distance between the windows is 2 mm. The optical depth, accounting for the refractive index is closer to 3 mm, although the optical depth of the far window decreases when the beam passes through large amounts of water.

Transmission OCT can be useful in making sensitive refractive index measurements because this approach does not depend on window reflections. The location of the strong signal in distance can be determined very accurately, and phase sensitive OCT techniques can make this measurement even more sensitive. The amplitude of the transmitted signal would be affected by particle density and by refraction of dissimilar liquids, such as water bubbles in the oil, which would act like lenses to divert and defocus the beams.

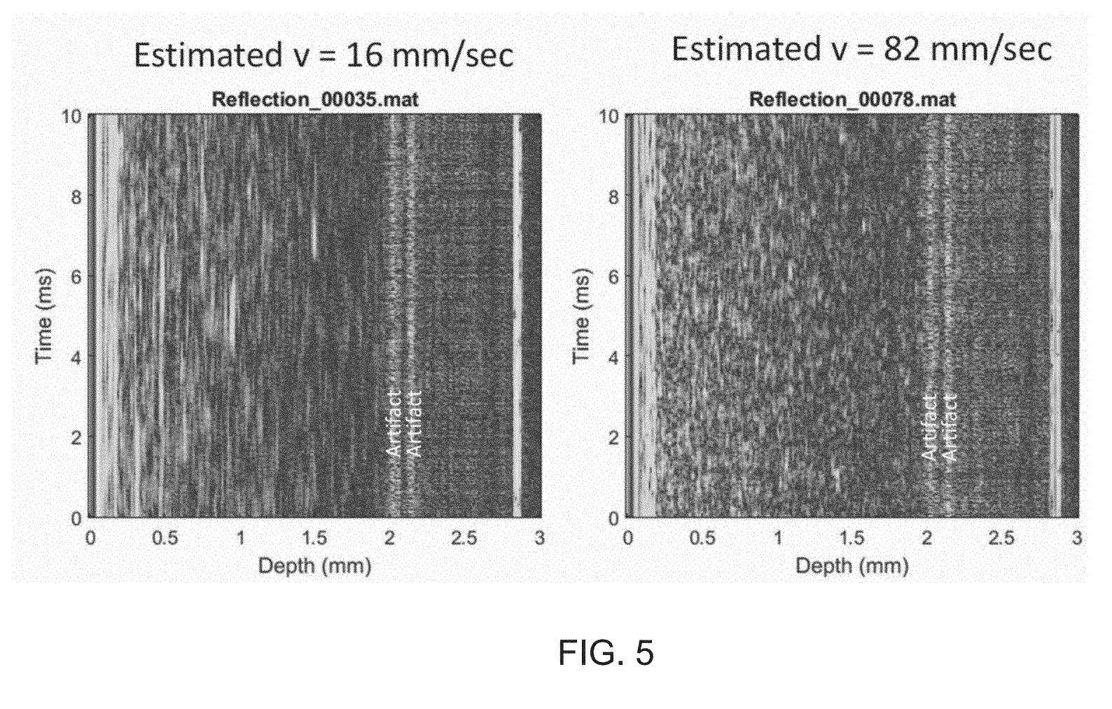

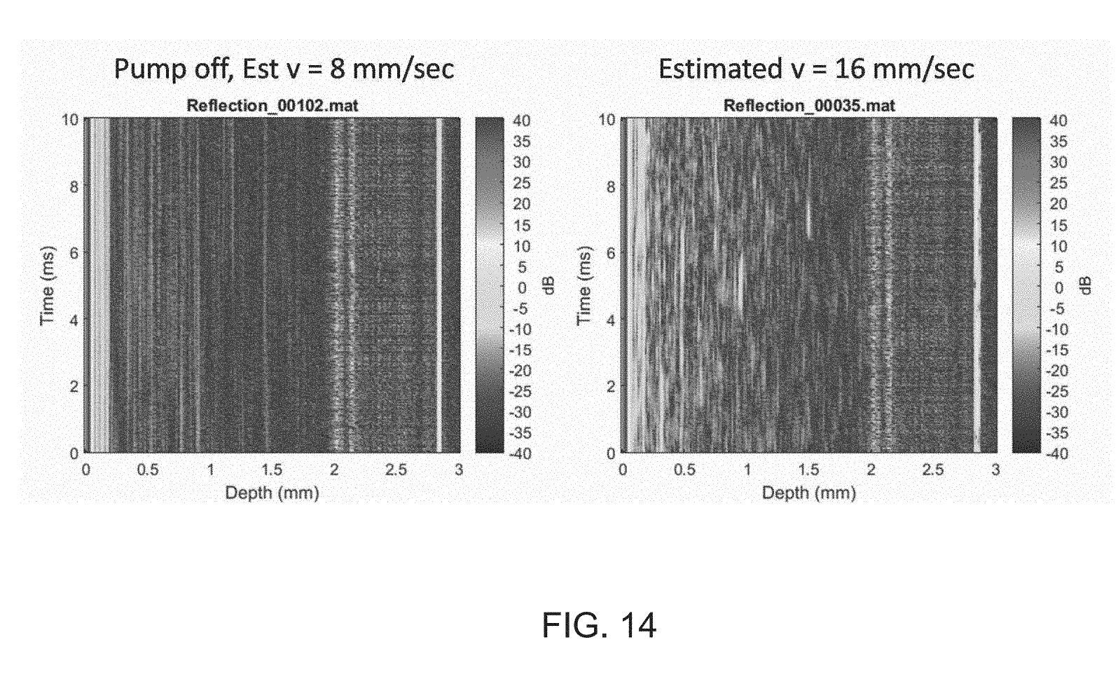

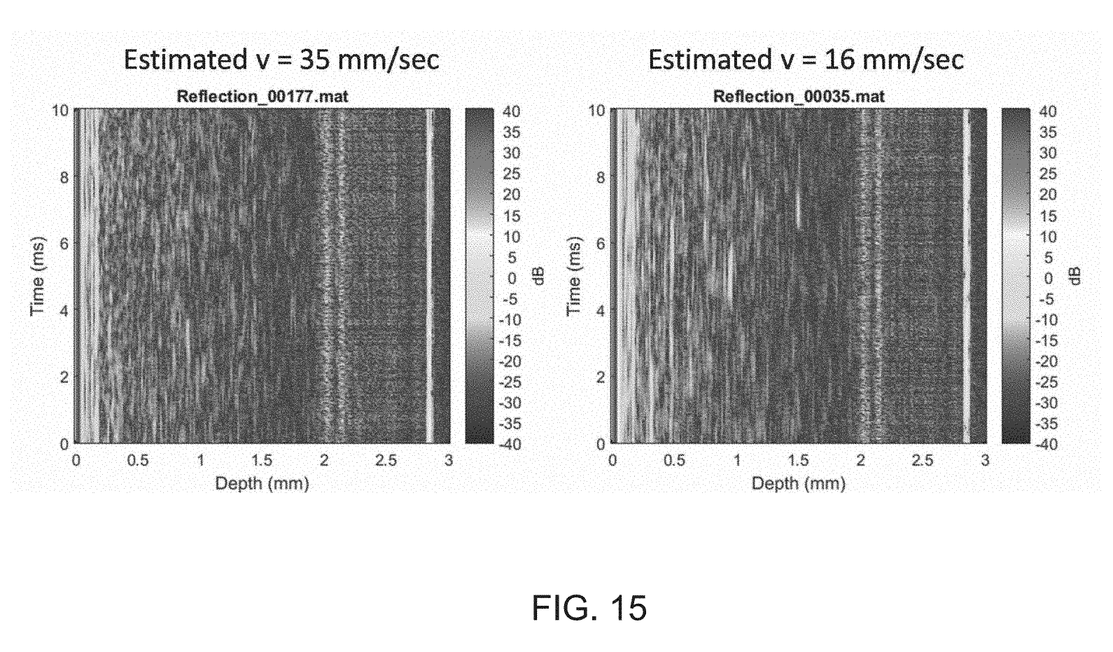

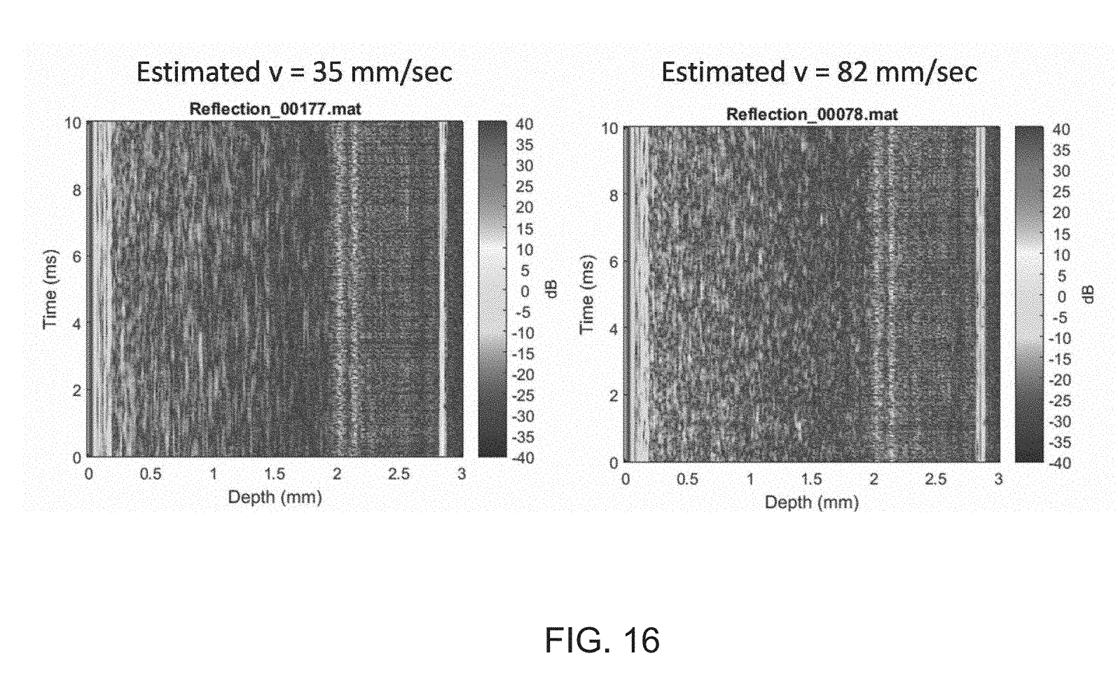

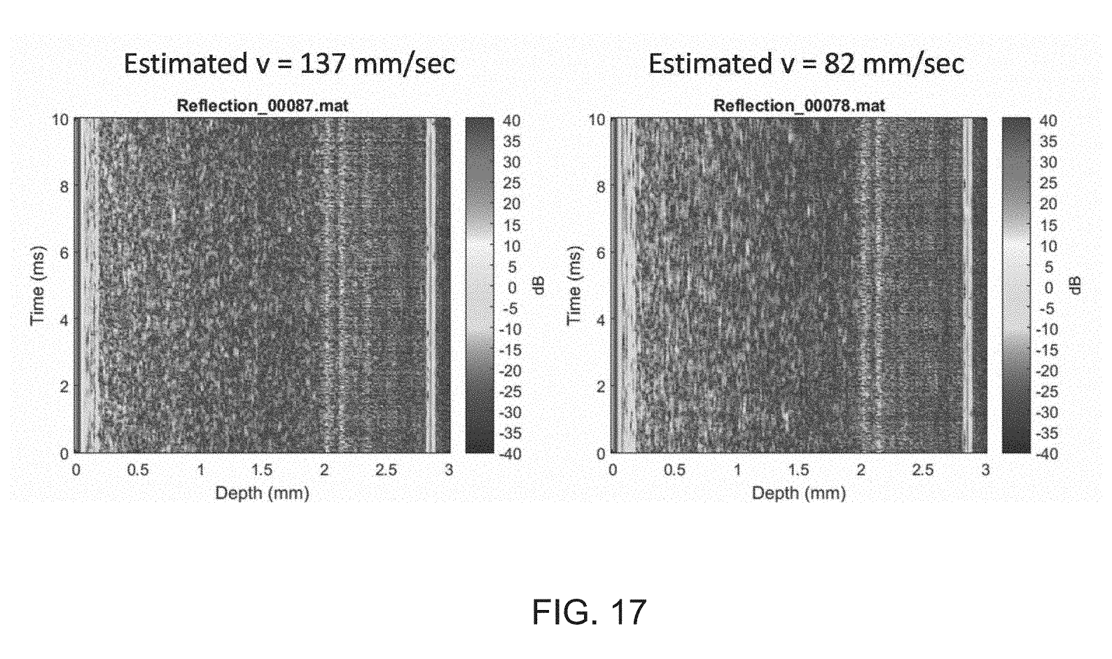

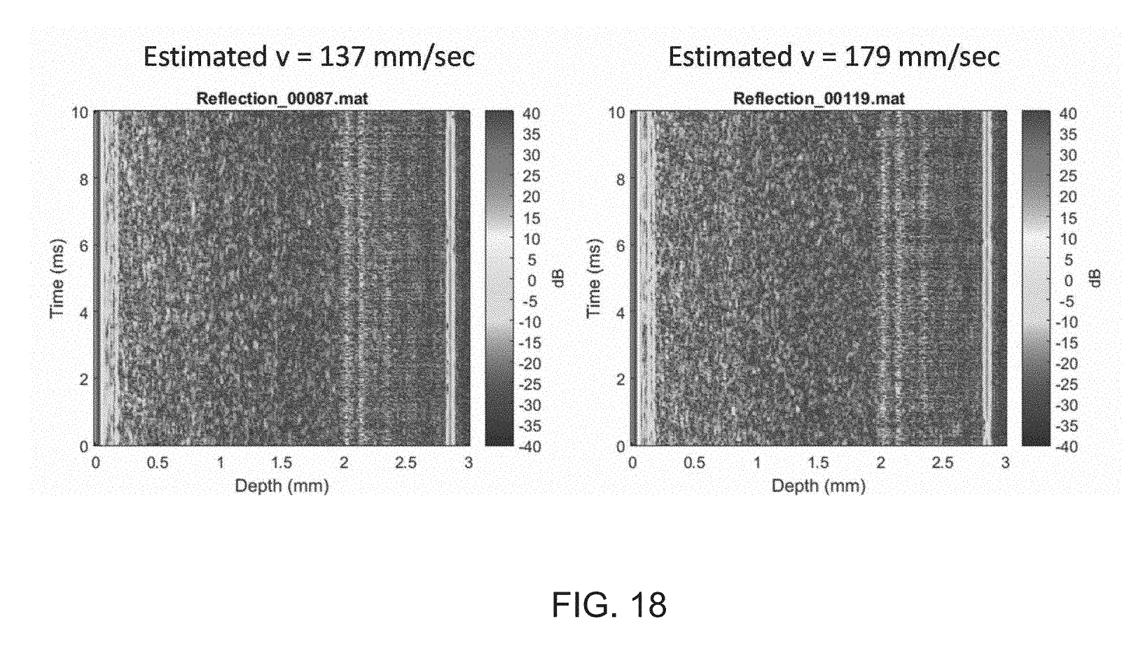

Flow velocity can be determined through an optical Doppler measurement, or through images analysis. The idea behind the image analysis is illustrated in FIG. 5, where the longer the particle streak, the slower the flow. More specifically, shown in FIG. 5 are two images at different flow rates. The velocity determines the streak length. The slower the flow, the longer the streak. The streak length in time equals the beam size divided by the flow rate. Thus, the estimated velocity for the conditions of the left hand image is 16 mm/sec, while the estimated velocity for the right hand image is 82 mm/sec. Further examples of side by side particle images obtained at different velocities are shown in FIGS. 14 to 18.

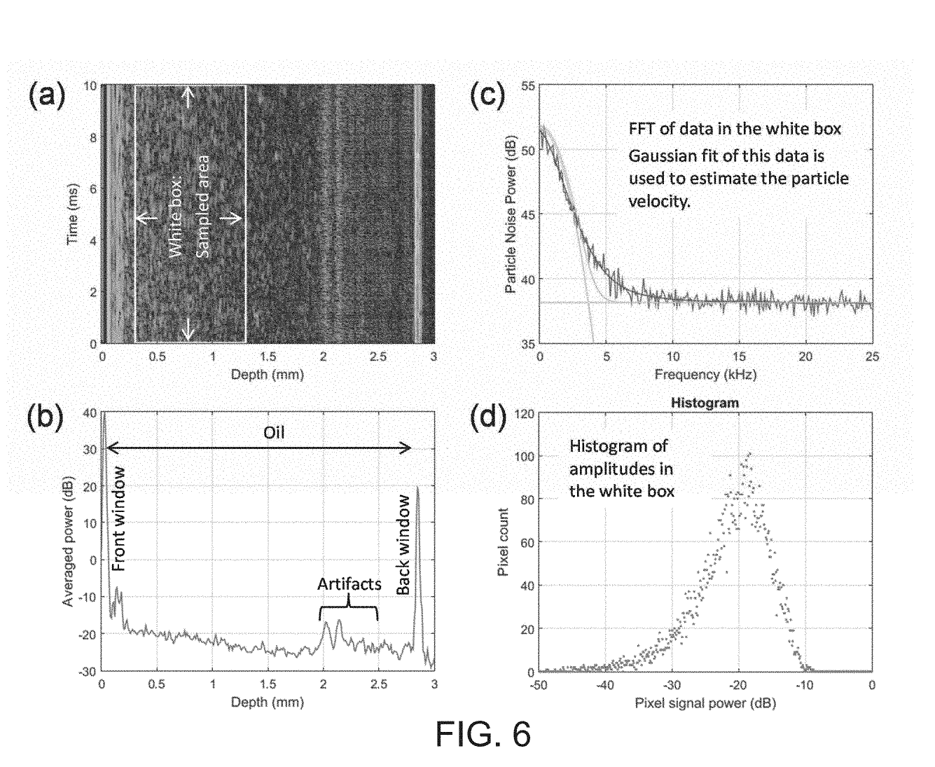

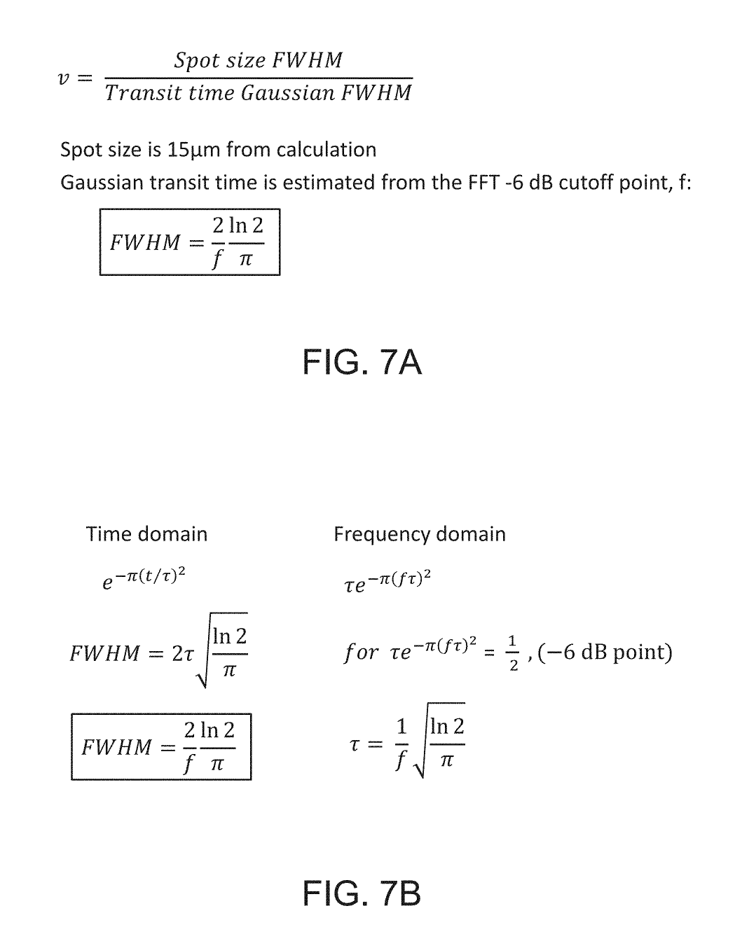

The image processing approach for flow velocity determination is further illustrated in FIG. 6. Instead of trying to isolate and measure individual streaks, many streaks are taken as an aggregate as outlined in the “white box” in shown FIG. 6a. The vertical streak at each pixel depth can be thought of a as noise signal with a certain bandwidth. The higher the bandwidth, the shorter the aggregate streaks, and the faster the flow. Each vertical line of pixels is Fourier transformed through FFT processing. The power spectrum of each vertical line of pixels is averaged with others within the white box to produce the Gaussian-like power spectrum seen in FIG. 6c. A Gaussian fit to the data results in a velocity estimate using the formulas shown in FIG. 7A. The Gaussian FT pair is shown in FIG. 7B.

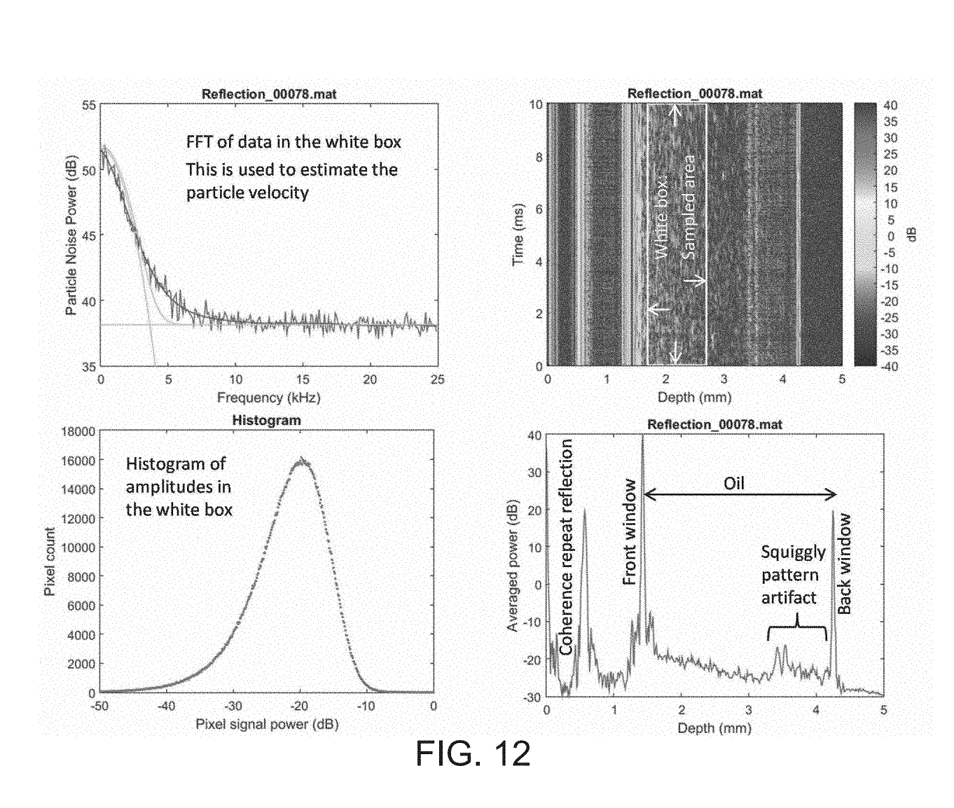

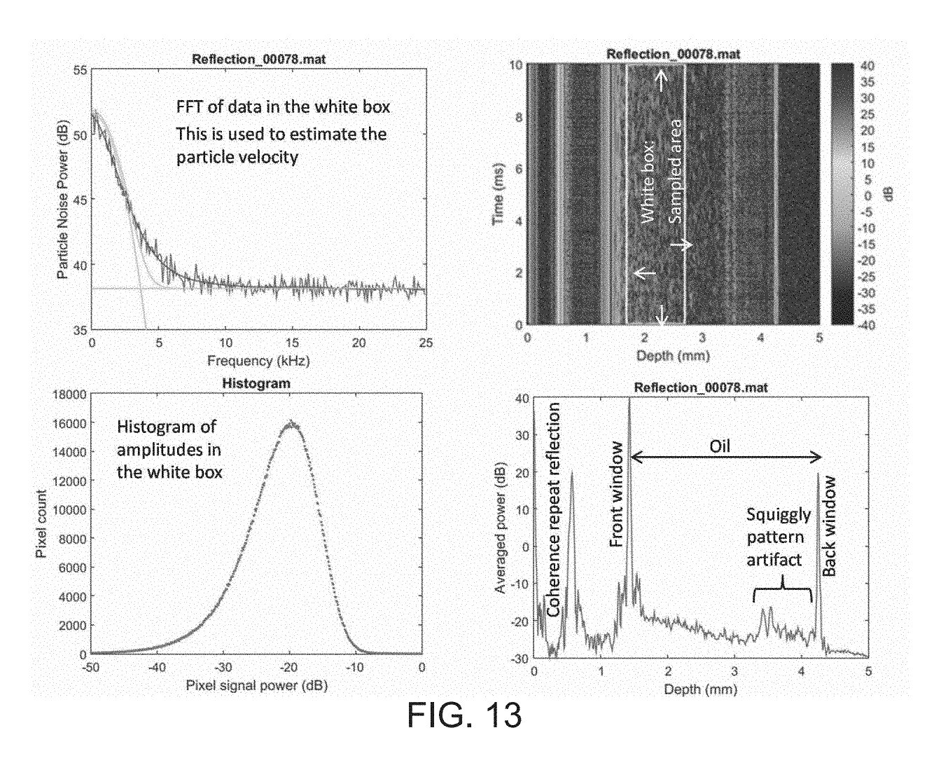

Additional examples of data processing steps used to determine particle velocities by image processing are presented in FIGS. 12 and 13.

The velocity estimate obtained through this method assumes scattering particles no larger than the beam size in the transverse direction. In a more general case the length of a vertical line of (high-signal) pixels depends on both the size of the particle in the transverse dimension (mapped as time in FIG. 5) as well as the speed at which it is traveling. With that in mind, this image processing method can provide one or more of the following:

an absolute velocity estimate assuming small (well-resolved transversely) particles;

an estimate of the relative particle size distribution assuming constant flow;

an estimate of velocity changes with time (e.g., between FIG. 5 left and FIG. 5 right) assuming constant particle size (distribution).

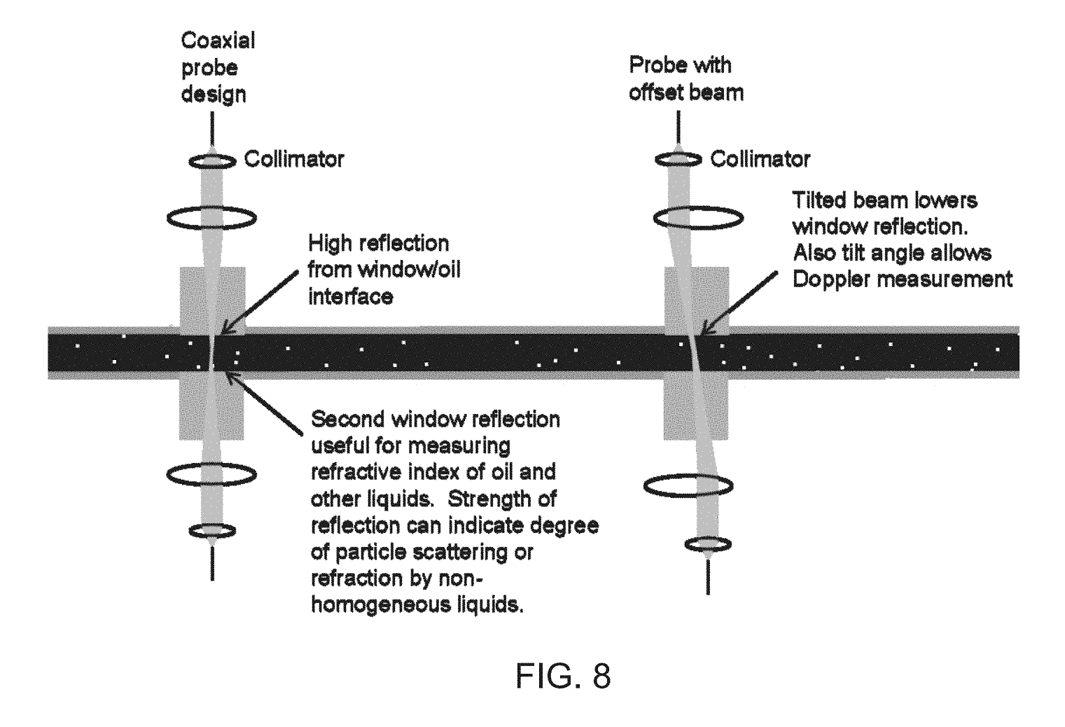

An alternate way of measuring flow velocity is by Doppler OCT [References 4,5]. To do this, the optical beam propagation direction cannot be perpendicular to the flow vector. This is because v_{measured}=v_{flow}×cos(θ) where θ is the angle between the beam and flow vectors. The offset beam probe shown on the right side of FIG. 8 allows for simultaneous Doppler measurements to be made with the particle images.

FIG. 8 presents side by side coaxial (left) and offset (right) probe designs for both reflection and transmission. The offset beam design is useful for obtaining high signal-to-noise. Very large samples powers can be applied without the window reflection saturating the detector in reflection mode. Another advantage is that the tilted beam allows Doppler measurements, because v_{measured}=v_{flow}×cos(θ) where θ is the angle between the beam and flow vectors.

The offset beam probe may be important for yet another reason. In cases in which the signal-to-noise ratio is not as high as desirable, boosting the optical power in the sample beam may result in improved sensitivity. Without an offset beam probe, the window reflected power becomes too great and saturates the detector. Thus the offset beam probe eliminates the problem.

Combining (multiplying) the transit time of the scattering particles (length of the vertical lines in FIGS. 5 and 6a) by the Doppler estimate of the particle speed provides an unambiguous estimate of the particle size in the transverse direction. This in turn allows an unambiguous estimate of the particle size in a given data frame and enables unambiguous estimates of particle size (distribution) changes with time. Particle size changes can be, for instance, indicative of aggregation, and/or precipitation, and/or contamination among others. In one example, the precipitation is from a dissimilar oil, e.g., asphaltene from an incompatible oil. Particle size distributions may also be serve as a “fingerprint” of the provenance of a particular sample, such as the location of the particular oil from which an oil sample was taken. The estimate of the transverse particle size may allow unambiguous estimation of the particle cross-section without the need for beam scanning.

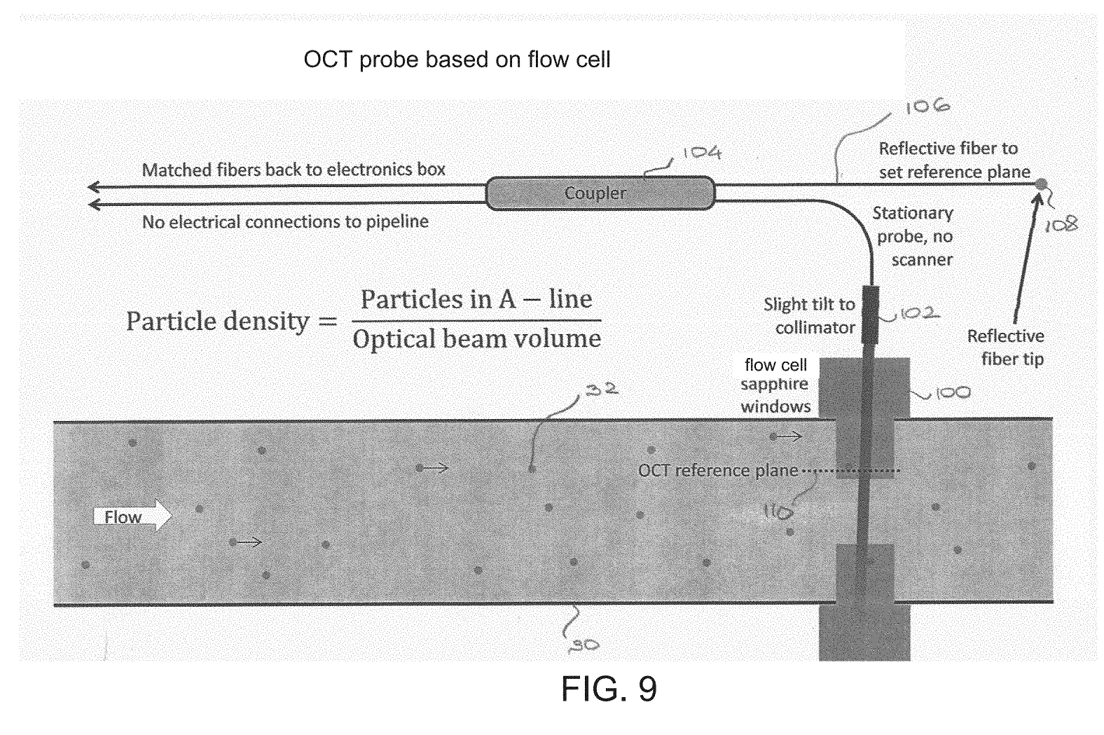

Further details regarding an OCT arrangement for conducting embodiments described herein are presented in FIG. 9. As seen in this figure, the medium measured is fluid 30, containing particles 32, essentially as described above. Cell 100 is a flow cell with sapphire windows. Collimator 102 is slightly tilted. The interference pattern is established between the input from the sample arm 104 and that from reference 106, employing reflective fiber, with reflective fiber tip 108 to set the reference plane. Dotted line 110 represents the OCT reference plane. From coupler 104 the matched fibers connect to suitable electronics. Using an arrangement such as that of FIG. 9, particle density can be determined by the equation: (Particles in A−Line)/Optical Beam Volume.

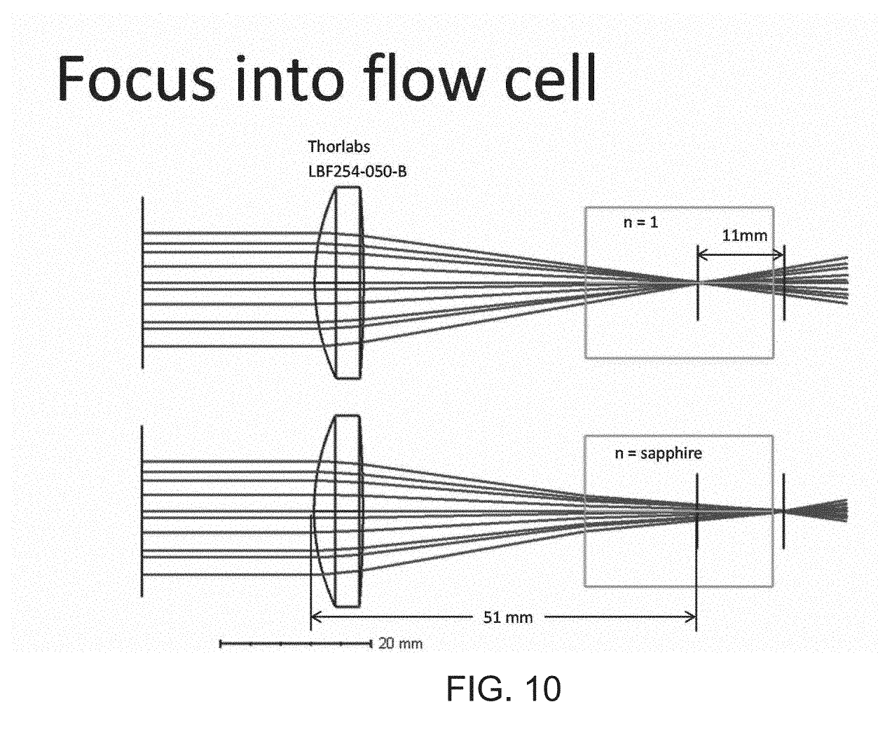

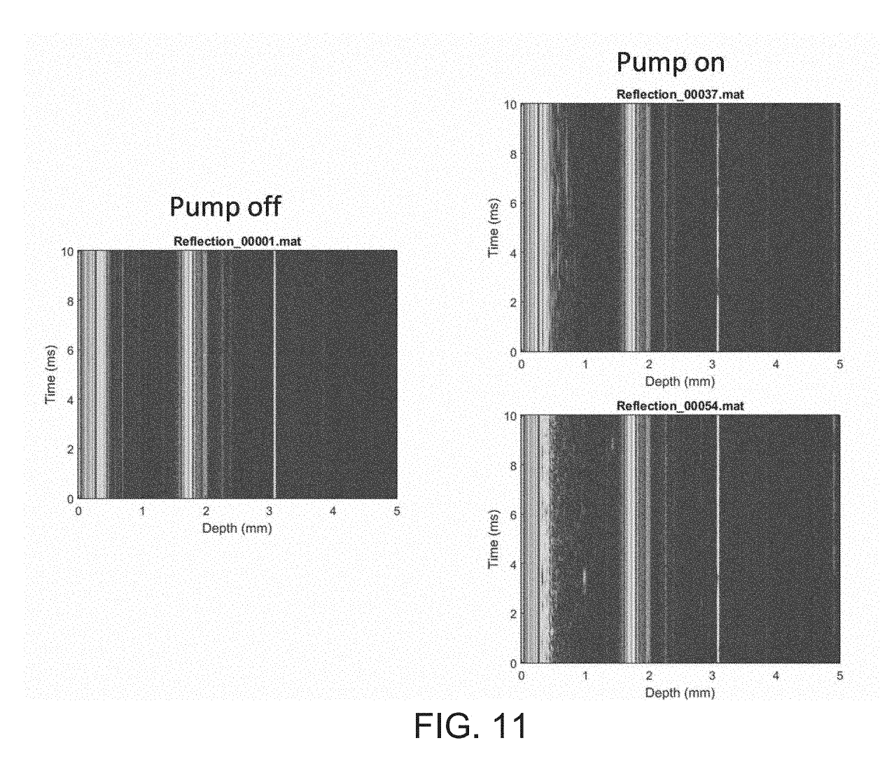

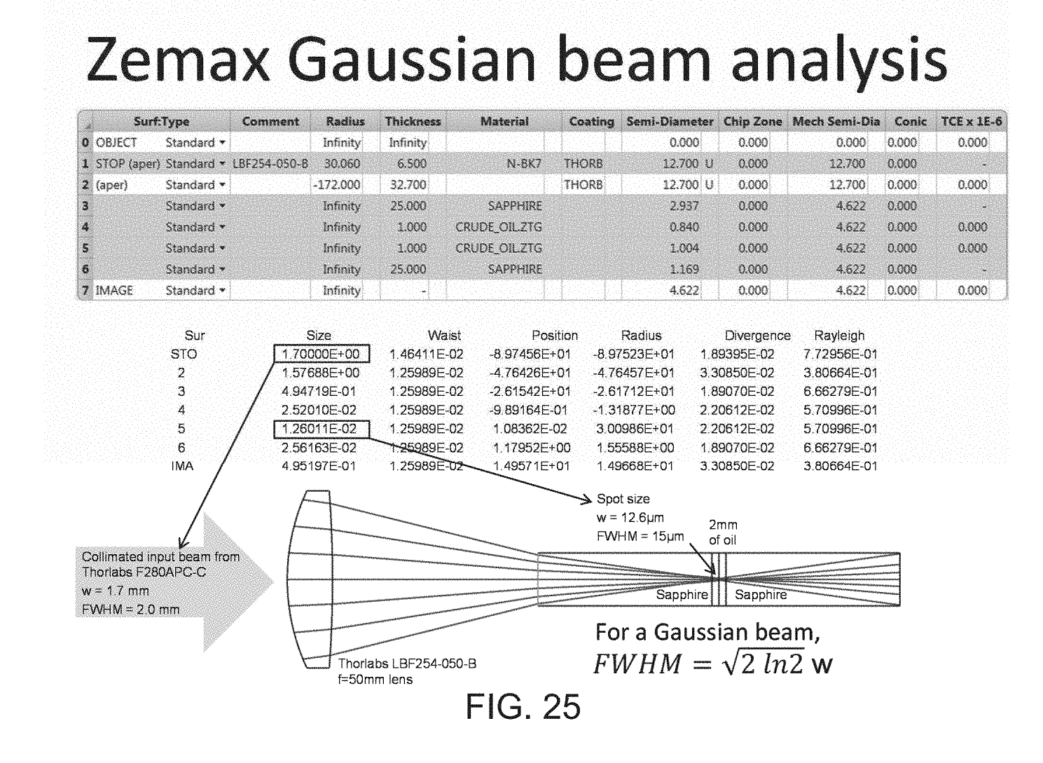

Focus into a flow cell is shown in FIG. 10. A Zemax Gaussian beam analysis is illustrated in FIG. 25.

One problem that may be encountered relates to the small scattering signal arising from the particles. In some cases, the problem can be observed even with a delivery of 20 mW or more to the oil cell. Higher numerical aperture (NA) collection values in the sample arm may address this problem (boost signal, reduce spot size in oil).

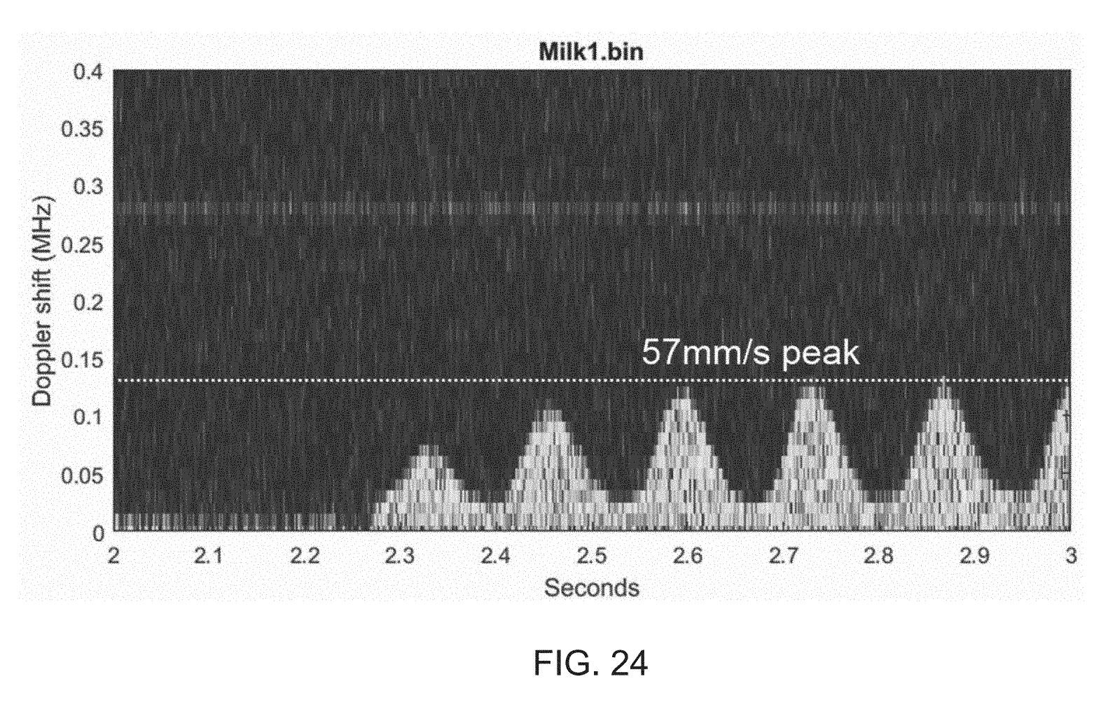

In a further embodiment of the invention, a peak velocity of 57 mm/sec was determined for milk with water using a Doppler probe, as shown in FIG. 24. Presumably the peristaltic pump flows are roughly the same for water/milk and the light West Texas Intermediate crude oil. However, the Doppler probe measured flow in a full ½ inch bore tube. The oil cell, with its 2 mm gap between windows, has a more restricted cross section, so larger flow rates would be expected.

In some cases, the SNR was found to be weak, sometimes close to the sensitivity of the OCT system. This can be addressed, for example, by increasing the optical power in the cell (above the 20 mW typically used), employing, e.g., a semiconductor optical amplifier. However, the reflectivity at the window/oil interface can be very high, 60 dB higher than the particulate signal in some cases. This reflectivity can be lowered by tilting the window, as described above. With the reduced reflectivity, the particulate signal can be boosted.

Experimental results showed that techniques described herein could be used to “see” particles in a 2 millimeter-wide stream of West Texas intermediate oil and in Cold Lake oil.

### REFERENCES

[1] M. R. Hee, J. A. Izatt, J. M. Jacobson, J. G. Fujimoto, and E. A. Swanson, “Femtosecond transillumination optical coherence tomography,” Optics Letters, 18 950 (1993);
[2] L. Li and L. V. Wang, “Optical coherence computed tomography”, Applied Physics Letters 91 141107 (2007);
[3] V. D. Nguyen, D. J. Faber, E. van der Pal, T. G. van Leeuwen, and J. Kalkman, “Dependent and multiple scattering in transmission and backscattering optical coherence tomography”, Optics Express, 21 29145 (2013);
[4] B. J. Vakoc, S. H. Yun, J. F. de Boer, G. J. Tearney, B. E. Bouma, “Phase-resolved optical frequency domain imaging”, Optics Express, 13 5483 (2005);
[5] I. Grulkowski, I. Gorczynska., M. Szkulmowski, D. Szlag, A. Szkulmowska R. A. Leitgeb, A. Kowalczyk, and M. Wojtkowski, “Scanning protocols dedicated to smart velocity ranging in Spectral OCT”, Optics Express, 17 23736 (2009).

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for analyzing a fluid sample, the method comprising:
flowing a sample through a conduit; and
obtaining OCT images of the sample in the conduit to determine at least one of: a density of particles present in the fluid sample; a particle flow velocity; a size of a particle in the sample; an index of refraction of the fluid; an index of refraction of an immiscible droplet in the fluid; and an image of an immiscible bubble in the fluid.

2. A system for analyzing a fluid sample, the system comprising:
a flow cell through with a sample flows; and
an optical coherence tomography system for obtaining images of the sample in the flow cell to determine at least one of: a density of particles present in the fluid sample; a particle flow velocity; a size of a particle in the sample; an index of refraction of the fluid; an index of refraction of an immiscible droplet in the fluid; and an image of an immiscible bubble in the fluid.
